(19) United States
(12) Patent Application Publication
(10) Pub. No.: US 2003/0109784 A1
(43) Pub. Date: Jun. 12, 2003

(54) Method of producing profiled sheets as prosthesis

(76) Inventors: Kwok Weng Leonard Loh, Singapore (SG); Teddy Eng Hoo Ong, Singapore (SG)

Correspondence Address: Lawrence N Ginsberg, 907 Citrus Place, Newport Beach, CA, 92660-3227, US

(21) Appl. No.: 10/275,144

(22) PCT Filed: Mar. 23, 2001
(86) PCT No.: PCT/SG01/00045

(30) Foreign Application Priority Data
May 10, 2000 (SG) 200002474-5

(51) Int. Cl. A61B 5/05

(52) U.S. Cl. 600/427

(57) ABSTRACT

A method of profiling a substrate as prosthesis for a structural defect in a patient whereby pressworking technique is used to press a prosthesis between a punch and a cavity mould to form the prescribed shape. The punch and cavity of the mould contains a profile that is computer generated and designed to closely match the patient's profile and to give the most natural and fitting prosthesis. The present method uses a set of 2-dimensional (2D) CT scans of the region around the defect and converts them into a 3 dimensional (3D) digital model, after which a prototype of the defective region is optionally produced by rapid prototyping techniques. The 3D digital model of the prosthesis is then used to digitally construct a set of profiling tools after which the actual punch and mould are physically produced.

### FIELD OF THE INVENTION

The present invention relates to prosthesis production. In particular, the present invention relates to the making of prostheses from data generated by computer tomography (CT) scanning.

### BACKGROUND OF THE INVENTION

Medical implants, such as titanium meshes, are often used for covering and protecting body tissues by securing onto bone structures with defects. In cranioplasty surgery, a missing patch of the skull is replaced by a prosthetic implant. Other defects include missing or deformed patches in limbs, hip and jaw. Conventional methods of fabricating the implants is by manual bending of the sheet to a shape estimated to be able to cover the missing or defective region based on x-ray data. The results are often inaccurate, requiring substantial manipulation by the surgeon during the actual implant operation. Traditional methods of manufacturing prosthesis are also plagued by inherent difficulties in quantifying and recording the modifications used to produce the prosthesis. Thus the quality of prostheses produced varies greatly.

The computerisation of contemporary manufacturing, together with computer-aided design (CAD) and computer aided engineering (CAE), has aided advances in prosthesis design and manufacturing in the medical field.

It is therefore an object of the present invention to provide an improved method for designing and fabricating prostheses.

### SUMMARY OF THE INVENTION

Accordingly, the present invention provides a method of profiling a substrate as prosthesis for a structural defect in a patient. The method employs a pressworking technique in which the substrate is pressed between a punch and a cavity mould to form the prescribed shape of the prosthesis. The punch and cavity of the mould contains a profile that is computer generated and designed to closely match the patient's profile and to give the most natural and fitting prosthesis. The present method uses a set of 2-dimensional (2D) CT scans of the region around the defect and converts them into a 3 dimensional (3D) digital model, after which a prototype of the defective region is optionally produced by rapid prototyping techniques. A 3D digital replacement of the defect is then generated and used to digitally construct a set of profiling tools after which the actual punch and mould are physically produced. The substrate, for example a titanium mesh, can then be pressed between the punch and mould to form the desired profiled prosthesis.

In the preferred embodiment, further touch-up can be optionally performed using the prototype of the defective region before the prosthesis is sent for sterilisation and implanting by a surgeon.

The present invention allows the fabrication of more accurate parts, and also has the advantage of being fast and less laborious. Furthermore, the digital data can be stored and compiled into a databank from which future designs may be sourced.

### BRIEF DESCRIPTION OF THE DRAWINGS

FIG. 1 is a flow diagram to show the preferred embodiment of the present invention.

FIG. 2 is a flow chart to show the results of the thresholding and is regiongrowing manipulations on the CT scan data.

FIG. 3 is a 3-dimensional (3D) model of a skull with a hole or missing patch as an example of a defective region.

FIGS. 4A and 4B are flow diagrams to illustrate two methods of creating a 3D digital replacement for a defect according to the present invention.

FIGS. 5A-5C shows the images produced for various steps of the mirroring technique.

FIGS. 6A and 6B are flow diagrams to show a method of creating the profiling tools according to the present invention.

FIG. 7 is a flow diagram to show the method of creating a 2D surface from a digital replacement according to step 105b.

### DESCRIPTION OF THE INVENTION

The following detailed description describes various embodiments for implementing the underlying principles of the present invention. One skilled in the art should understand, however, that the following description is meant to be illustrative of the present invention, and should not be construed as limiting the principles discussed herein. As one skilled in the art will appreciate, there may be different software capable of achieving the steps described. The specific examples and software described are used as examples only. In the following discussion, and in the claims the terms “including”, “having” and “comprising” are used in an open-ended fashion, and thus should be interpreted to mean “including but not limited to . . . ”. The term “defect” is used in a generic sense to refer to any undesirable area or patch that needs replacement, covering or reinforcement, including, but not limited to, hole, fractures and deformed structures, particularly bone structures, such as the jaw, limb, hip or skull. The term “prosthesis” is used in a general sense to refer to any artificial structures that are fabricated using the present invention for replacement, reinforcement or cosmetic purposes. For example, the prosthesis may be a reinforcement link that can be bolted onto the two sides of a fracture, or metallic profiles that can be added onto the surfaces of bones to alter the appearance of the relevant part of the body.

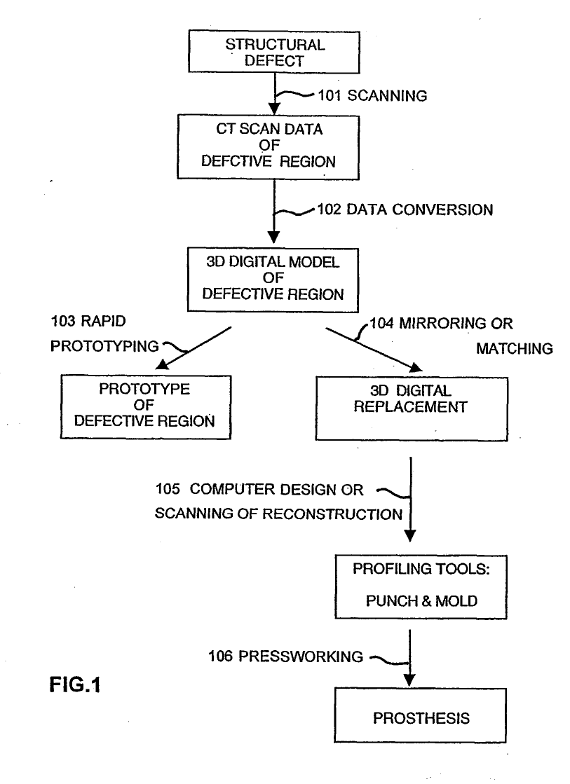

FIG. 1 shows a general method of profiling and producing a prosthesis according to the present invention. Each of these steps (101 to 106) will be described in detail below. For ease of explanation, a skull with a hole or missing patch will be used as an example to explain the method.

The first step 101 is to generate the CT scan data of the patient around the region of the defect. The parameters of the CT scanning procedure, together with the scan data, are saved into a computer. The CT data format is converted to generic image format using, for example, Interactive Medical Image Control System (MIMICS) software from Materialise, Belgium. This allows the visualization and segmentation of the CT images and also the generation of coloured 3D models of the defective region.

In order to define exactly the object to be visualized or produced in 3D (step 102), segmentation of the CT scan data is required. In this case, the defective region is the top half of the skull that has a hole due to missing bone tissue, and the object to be visualised is the surrounding bone structure. Using the MIMICS software, 3 steps are generally performed (1) thresholding; (2) region growing and (3) manual editing.

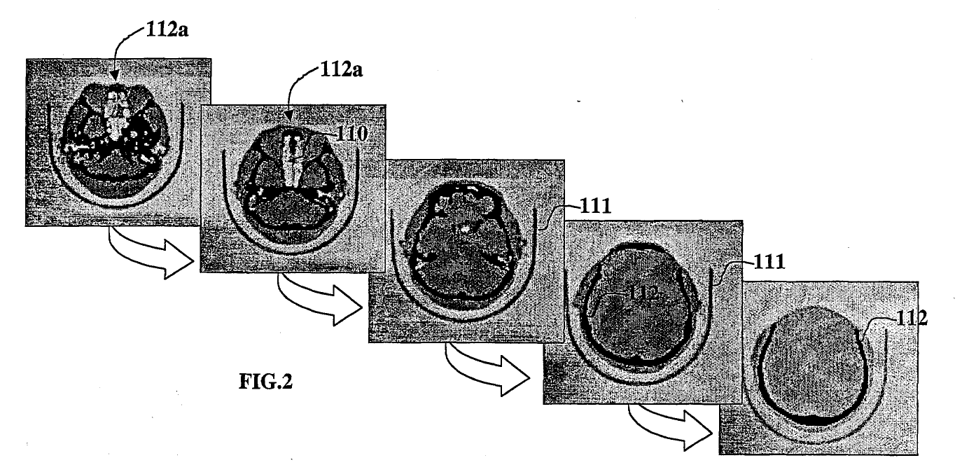

The thresholding technique is shown in greater detail in FIG. 2. This technique exploits the differences in density of different tissues to select image pixels with a higher or equal value to the prescribed threshold value. Since bone tissue has higher density than brain matter 110, muscles or skin etc., the bone tissue 112 can be sequentially selected. In FIG. 2, the head 112a is positioned above a supporting device 111.

The regiongrowing technique is used after thresholding to isolate the area which has the same density range but are not related to the bone tissue under study.

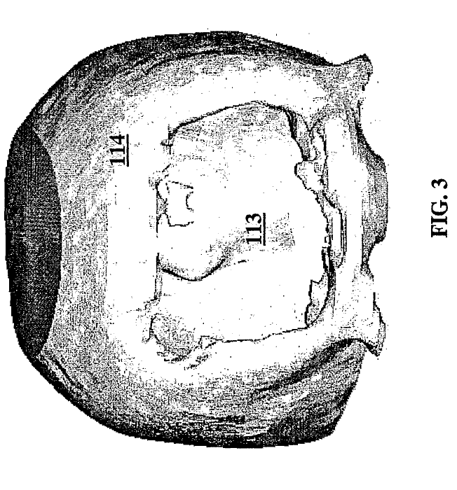

Manual editing is used to perform local corrections and to remove noise from the segmented object. The image is then converted into a 3D CAD model of the defective region, as shown in FIG. 3. In this figure, the defect is an anterior hole 113 of a skull 114, and the defective region is the top half of the defective skull. A suitable software, such as the CT-Modeller Program from Materialise, is used to generate the STL model from the 3D Medical image constructed earlier using the MIMICS module. A prototype or physical model of the defective region can then be produced using a rapid prototyping machine that accepts digital data in STL format (step 103).

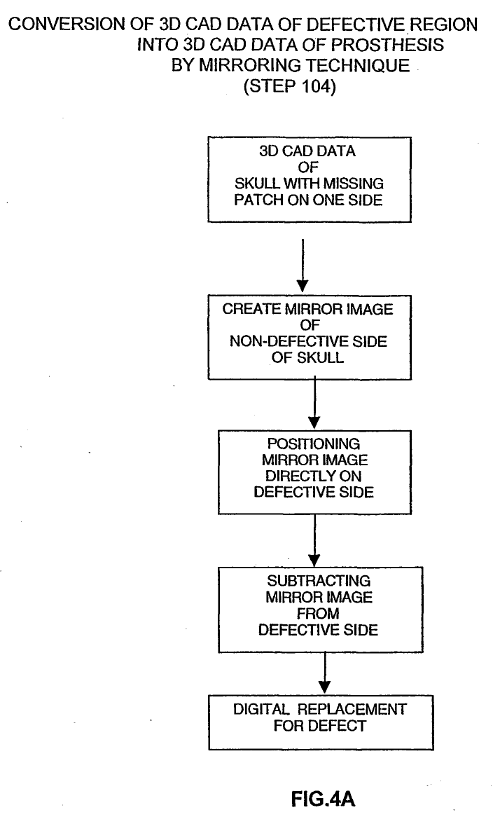

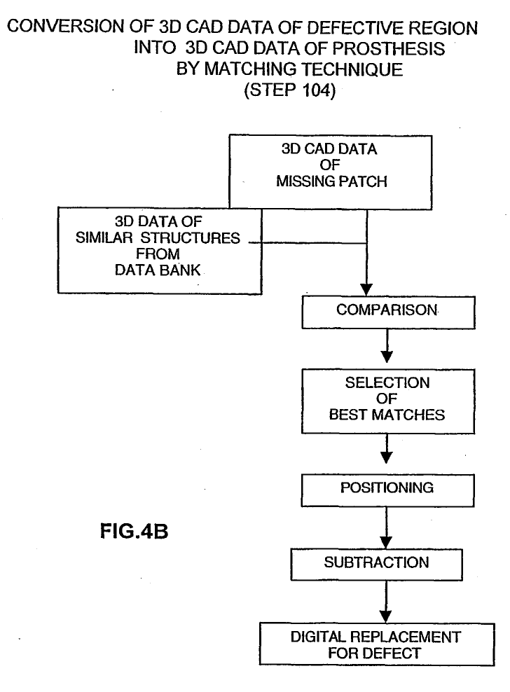

Step 104 of FIG. 1 is the generation of the 3D (CAD data) digital replacement for the defect. This step first requires the generation of the 3D digital data of the defect (in this example, it is a patch that closes up the hole). There are two examples of how this 3D digital replacement may be obtained, as shown in FIGS. 4A and 4B.

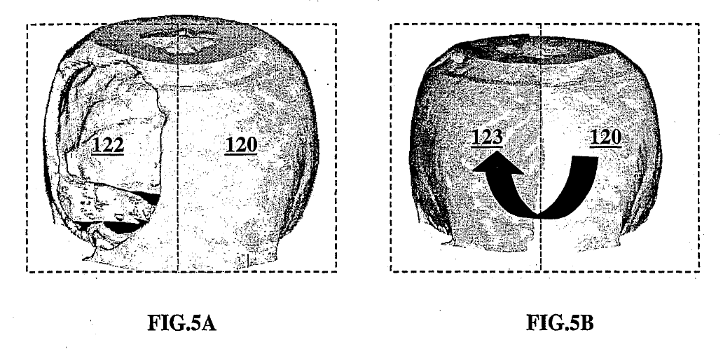

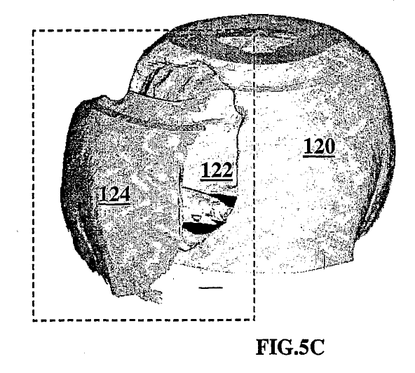

Referring first to FIGS. 4A and 5A-5C, a mirroring technique may be used if the defect is on one side of a bone structure that has a natural symmetry. For example, the defect is a hole on one side of the skull, and the other side of the skull constitutes part of the 3D CAD data of the defective region. This mirroring technique isolates and copies the non-defective half of the skull 120 and repositions a mirror image 123 of the copy onto the defective half 122 as shown in FIG. 5B. Subtraction is then performed on the repositioned mirror image from the defective side of the original skull to obtain the 3D digital replacement 124 for the hole. Any excess portions may be removed and errors corrected.

Referring now to FIG. 4B, the matching technique may be used instead of the mirroring technique to obtain the digital replacement. This technique is most useful for replacement of missing patches that do not have any available non-defective counterpart within the CT scan data of the patient. For example, anterior and posterior cranial defects cannot be replaced by the mirroring technique. In the matching technique, the 3D CAD data or CT scan data from other normal people are collected and stored in a databank. A search is then conducted on the databank to find a suitable match as a reference. The reference skull is then repositioned, superimposed and subtracted against the defective skull to obtain the digital replacement. If the reference skull itself has holes or other defects (such as complete matching problems) and cannot effectively cover the defect, superposition may be performed on multiple reference skulls to create a suitable digital replacement. The union of all the copied images can then be obtained and subtracted from the original defective skull to obtain the digital replacement. Any excess or unwanted portions is manually removed.

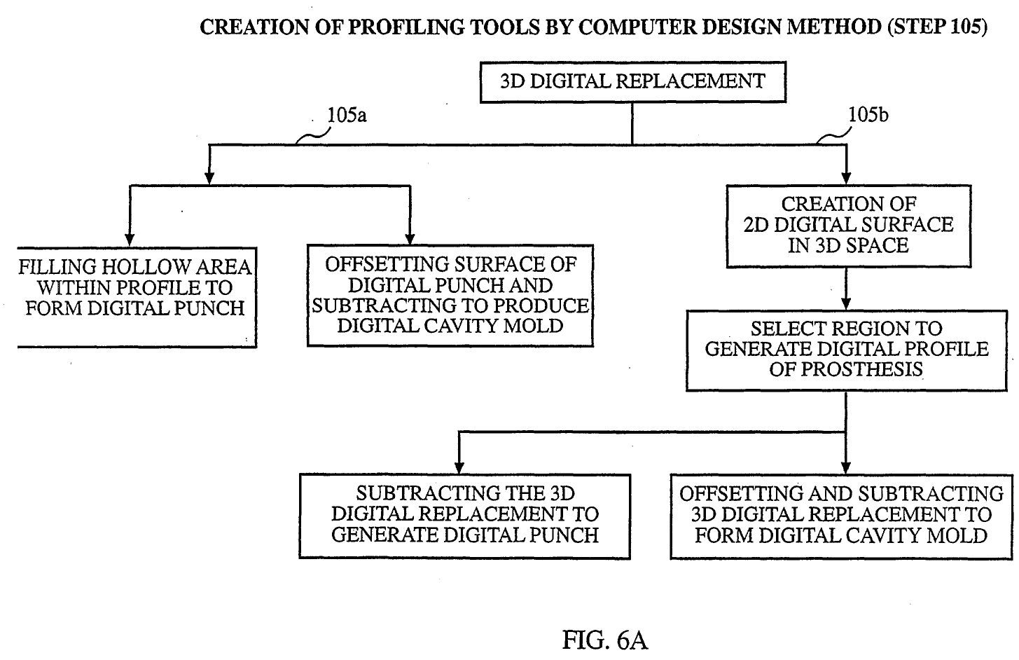

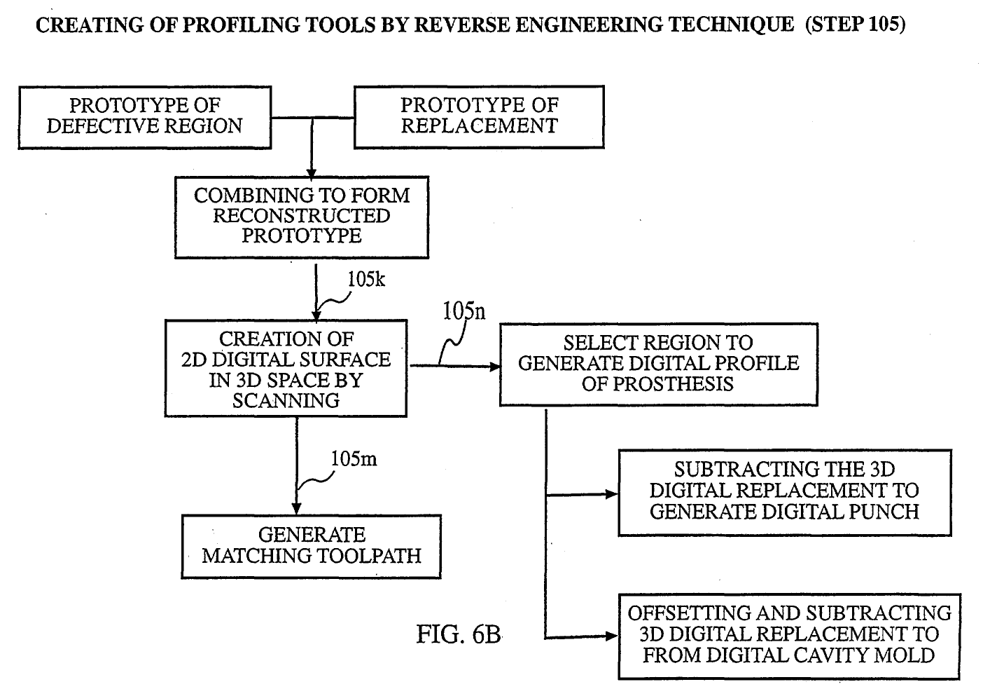

Step 105 in FIG. 1 involves the making of the actual profiling tools from the 3D digital replacement. Two methods for doing so are shown in FIGS. 6A and 6B.

In the embodiment shown in solid arrows in FIG. 6A, the digital punch and cavity mould are completely computer designed in stereolithography (STL) format, without the need to fabricate any actual prototypes. In this computer design method, the punch and cavity mould are designed with reference to the digital replacement created from the aforementioned methods using, as an example, the Surfacer software from Imageware, USA.

In the method shown in path 105a, the digital punch is created by first adding a boundary allowance of, for example, 10-15 mm to the edge or boundary of the digital replacement. The dimensions of the boundary allowance may be determined according to the needs of the users. Since the digital replacement is typically in the shape of a shell, e.g. a portion of a skull, the hollow part of the shell is digitally filled and a holder added to create the digital punch in the computer. To create the digital cavity mould with the appropriate profile from path 105a, the digital punch is offset by an appropriate thickness to cater for the thickness of the substrate during pressworking. For example, for a titanium mesh plate of 0.5 mm thickness, the profile of the punch is offset by 0.5 mm. A solid block is then created and the 0.5 mm-enlarged digital punch subtracted therefrom to create a cavity in the solid block. The digital cavity mould is created after adding slots on the mould for locating purposes during pressworking on the press machine. The punch and mould are then physically fabricated using rapid prototyping techniques, for example, selective laser sintering (SLS).

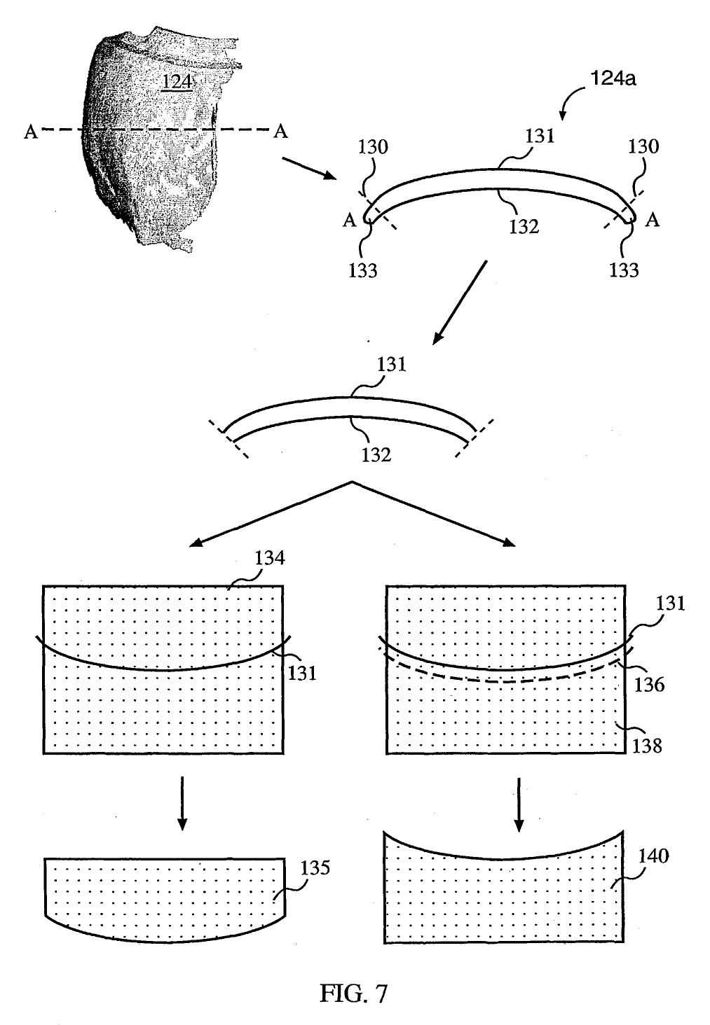

An alternative computer design method as shown by path 105b in FIG. 6A, and illustrated in greater detail in FIG. 7. In this technique, a cross-section 124a along line A-A is made across the digital replacement of the defect 124. The points 133 connecting the upper and lower surfaces of the 3D digital replacement are removed beyond locations 130. The remaining points can be divided into 2 subsets: one subset representing the upper surface 131 of the 3D digital replacement, the other subset representing the lower surface 132 of the same. A 2D surface may be generated from one subset by separating the two surfaces. This is done by selecting the points that are within a certain maximum distance apart. Those that are farther away (i.e. those that represent the non-selected surface) are removed. In the example shown in FIG. 7, the points on the upper surface 131 are selected and the points on the lower surface 132 are removed. A digital punch 135 can then be created by introducing a digital solid block 134, and subtracting the 2D upper surface 131 from the digital solid block 134. The cavity mould is generated by first offsetting surface 131 by a small distance to create profile 136 to account for the thickness of the prosthesis. Another digital block 138, is then created and subtracted with surface 136 to create the cavity mould 140.

Referring now to FIG. 6B, this embodiment uses reverse engineering techniques that requires the making of the physical prototypes of the defective region (referred to as defective region prototype) and the replacement part (referred to as replacement prototype). These physical prototypes may be fabricated with a rapid prototyping machine using the 3D CAD data of the defective region (a hole in the skull in the example given) and the 3D digital replacement in STL format (the replacement part for the hole in the example given) obtained by the methods as described above. Once the prototype of the defective region and the replacement part are obtained they are fitted together to form a reconstructed prototype (for example, a reconstruction of the skull of the patient). The reconstructed prototype is then scanned into a computer to form a set of 3D points of the reconstruction. The scanning may be performed using a laser digitiser, e.g. Mercury from Matuo, Japan. A 2D surface can then be reconstructed from the set of points. The digital punch and digital cavity mould, followed by the physical punch and cavity mould, can then be created by various methods. In one method, the 2D surface is used to generate a tool path for a machining process, as shown in path 105m e.g. using Unigraphics software. The mould and punch are then fabricated by conventional machining methods such as high speed computer numerical control milling. In an alternative method of generating the digital punch and digital cavity mold is shown in step 105n of FIG. 6B. This is the same technique as described for step 105b and FIG. 6A, whereby a digital punch is created by introducing a digital solid block, and subtracting the 2D surface from the digital solid block. The cavity mould is generated by offsetting the profile of the punch by a small distance to account for the thickness of the prosthesis, and subtracting with the mould to create the cavity mould.

The actual prosthesis is fabricated by pressworking techniques using, for example, a press machine in which a substrate is pressed between the punch and cavity mould to create the desired profile. The substrate may be any material required by the user, but is typically biocompatible, of high impact strength and non-biodegradable for a permanent structural prosthesis. For cranioplasty, a typical prosthesis used is a titanium mesh which needs an area slightly larger than the surface area of the defect. The extra area (i.e. the boundary allowance) is used for the placement of screws and other attachment devices during surgery. After pressworking, the prosthesis may be checked against the prototype of the defective region, and touching up and trimming may be performed to give the best fit.

Other prosthesis that can be fabricated using the present invention includes titanium or other metal links that are used to support a fractured bone structure. For example, a fractured hip may be reinforced for quicker recovery by providing a metallic link that is screwed to the two sides of the fractured bone structure. Using the present invention, an accurate profile of the link may be shaped, and secured onto the patient fittingly. Another application is in cosmetic surgery, such as jaw profile modification. In this example, the prosthesis would be secured onto the jawbone, either as a replacement of a defect or a missing patch, or simply to give an improved and desired check profile.

While the present invention has been described particularly with references to cranioplasty, it should be understood that the examples are for illustration only and should not be taken as limitation on the invention. In addition is clear that the method and apparatus of the present invention has utility in many applications where material shaping is required. It is contemplated that many changes and modifications may be made by one of ordinary skill in the art without departing from the spirit and the scope of the invention described.

1. A method of profiling a substrate as a prosthesis for a structural defect in a patient comprising:
a) generating CT scan data of a defective region of said patient in the region around said structural defect;
b) converting said CT scan data of said defective region into a 3-dimensional digital model of said defective region;
c) fabricating a defective region prototype using said 3-dimensional digital model of said defective region;
d) creating a 3-dimensional digital replacement of said structural defect, steps comprising:
(i) obtaining a non-defective 3-dimensional digital model of at least one non-defective subject;
(ii) comparing said non-defective 3-dimensional digital model with said 3-dimensional digital model of said defective region;
(iii) selecting said non-defective 3-dimensional digital model having a non-defective area that matches a defective area in said 3-dimensional digital model of defective region; and
(iv) subtracting said non-defective 3-dimensional model from said 3-dimensional digital model of said defective region to produce said 3-dimensional digital replacement;
e) fabricating a set of profiling tools; and
f) pressing said substrate with said profiling tools to form said prosthesis.

2. A method according to claim 1 further comprising the step of fabricating a replacement prototype from said 3-dimensional digital replacement; and
combining said replacement prototype and said defective region prototype to form a reconstruction prototype before step (e).

3. A method according to claim 2 wherein said step (e) further comprises the steps of:
(i) scanning said reconstruction prototype to create a 2-dimensional digital surface;
(iii) selecting a region of said 2-dimensional digital surface corresponding to said defective region to generate a digital profile; and
(iv) fabricating a set of profiling tools using said digital profile.

4. A method according to claim 3 wherein said step (iv) further comprises the steps of:
producing a digital punch and a digital mould using said digital profile; and
fabricating a set of profiling tools from said digital punch and said digital mould.

5. A method according to claim 3 wherein said step (iv) further comprises the steps of:
generating a tool path for a high speed milling machine: and
milling a mould cavity and a punch surface using said high speed milling machine.

6. A method according to claim 1 wherein step (e) further comprises:
(i) creating a digital block
(ii) offsetting and subtracting a prescribed region of said digital replacement from said digital block to create a digital cavity mould;
(iii) filling the hollow area within said digital replacement to form a digital punch; and
(iv) fabricating a mould and a punch from said digital mould and digital punch respectively.

7. A method according to claim 6 wherein a 2-dimensional surface of said digital replacement is first created before step (ii); and steps (ii) and (iii) are performed using said 2-dimensional surface instead of said digital replacement.

8. A method according to claim 1 wherein said structural defect comprising of said defective area is located on a defective side opposite to a non-defective side of a substantially symmetrical bone structure; and step (d) further comprises:
(i) creating a mirror image of said non-defective side of said bone structure;
(ii) positioning said mirror image directly on said defective area; and
(iii) subtracting said mirror image from said defective side to produce said digital replacement.
